(19) United States
(12) Patent Application Publication
(10) Pub. No.: US 2023/0033627 A1
(43) Pub. Date: Feb. 2, 2023

(54) SELF-SEALING FRAGRANCE DISPENSER

(71) Applicant: GARRETT LYNN DELISLE, LEHIGH ACRES, FL (US)

(72) Inventor: GARRETT LYNN DELISLE, LEHIGH ACRES, FL (US)

(21) Appl. No.: 17/390,983

(22) Filed: Aug. 1, 2021

(51) Int. Cl. A61L 9/12

(52) U.S. Cl. CPC A61L 9/12; A61L 2209/134

(57) ABSTRACT

A self-sealing fragrance dispenser can be positioned within an air channel having active and inactive air flow states. The dispenser can include a main body having an cavity in which a fragrance medium can be disposed, an access door, an intake door, and an exhaust door. The intake and exhaust doors can be biased in closed positions via bias structures or gravity. Pressures exerted against the intake and exhaust doors when the air channel is in the active air flow state place the intake and exhaust doors in respective closed intake and closed exhaust door states that seal the amount of fragrance medium within the cavity when the air channel is in the inactive air flow state.

### FIELD OF THE INVENTION

The present invention relates to fragrance dispensers.

### BACKGROUND OF THE INVENTION

Fragrance dispensers are devices that contain an amount of fragrance media, which release a fragrance into the air surrounding the dispenser. Fragrance media can be provided as a solid or liquid.

### SUMMARY OF THE INVENTION

It is an object of the present invention to provide a self-sealing fragrance dispenser.

It is another object of the present invention to provide a self-sealing fragrance dispenser that includes functional utility.

The present invention provides a self-sealing fragrance dispenser configured for positioning within an air channel having an active and inactive air flow states.

In an exemplary embodiment of the present invention, a self-sealing fragrance dispenser can include a main body formed of non-porous material defining a cavity configured to surround a fragrance medium.

In an exemplary aspect, such a main body can include an access door moveably engaged with the main body, with the access door being configured to have an access door open position to allow removable placement of the fragrance medium within the cavity and an access door closed position.

In another exemplary aspect, such a main body can further include an intake door moveably engaged with the main body, with the intake door being biased in an intake door closed position by an intake door bias force and being moveable to an intake door open position that exposes the cavity.

In a further exemplary aspect, such a main body can further include an exhaust door moveably engaged with the main body, with the exhaust door being biased in an exhaust door closed position by an exhaust door bias force and being moveable to an exhaust door open position that exposes the cavity.

In another exemplary aspect, the intake and exhaust door bias forces, which can be provided by a bias structure or gravity, can be configured place the intake and exhaust doors in respective closed intake and closed exhaust door states when the air channel is in the inactive air flow state, so as to seal the fragrance medium within the cavity.

In still a further exemplary aspect, the intake and exhaust door bias forces can be configured to be less than respective pressures exerted against the intake and exhaust doors when the air channel is in the active air flow state. Accordingly, when the air channel is in the active air flow state, the forced air can move through and/or across the fragrance medium and carry the scent along its way.

In a further exemplary aspect, one or more of the intake door bias force and the exhaust door bias force can be provided by a bias structure spring or gravity.

In still another exemplary aspect, the intake, exhaust, and access doors can cooperatively provide an air tight seal around the cavity.

In still a further exemplary aspect, the main body can include at least one mounting flange configured to mount the main body to a structure defining the air channel.

In another exemplary aspect, the main body can include at least one fixation structure positioned within the cavity and configured to fix the fragrance medium within the cavity.

### BRIEF DESCRIPTION OF THE DRAWINGS

FIG. 1 illustrates an exemplary embodiment of a self-sealing fragrance dispenser having a main body, an intake door, and an exhaust door.

FIG. 2A illustrates an exemplary hinge structure and spring cooperatively providing moveable engagement of an intake door having a biased closed position and an open position.

FIG. 2B illustrates an exemplary hinge structure and spring cooperatively providing moveable engagement of an exhaust door having a biased closed position and an open position.

FIG. 2C illustrates an exemplary hinge structure provided as a pivot rod and supporting bracket.

FIG. 3 illustrates an exemplary dispenser having an optionally skewed main body to facilitate utilization of gravity-based bias forces.

FIG. 4A illustrates an exemplary access door in a closed position.

FIG. 4B illustrates an exemplary access door in an open position.

FIG. 4C illustrates an another exemplary access door in a closed position.

### DETAILED DESCRIPTION

It is an object of the present invention to provide a self-sealing fragrance dispenser.

It is an object of the present invention to provide a self-sealing fragrance dispenser that can be single- or multi-use.

It should be noted that this disclosure includes a plurality of embodiments each having a plurality of elements, aspects, and/or steps and such elements, aspects, and/or steps need not necessarily be interpreted as being conjunctively required by one or more embodiments of the present invention. In particular, all combinations of such elements, aspects, and/or steps can enable a separate embodiment of the present invention, which may be claimed with particularity in any future filed Patent Application. Moreover, such elements, aspects, and/or steps disclosed herein, whether expressly or implicitly, are to be construed strictly as illustrative and enabling, and not necessarily limiting. Therefore, it is expressly set forth that any elements, aspects, and/or steps, independently or in any combination of one of more thereof are merely illustratively representative of one or more embodiments of the present invention and are not to be construed as necessary in a strict sense.

Further, to the extent the same element, aspect, or step is defined differently anywhere within this disclosure, whether expressly or implicitly, the broader definition is to take absolute precedence, with the distinctions encompassed by the narrower definition to be strictly construed as optional.

Illustratively, perceived benefits of the present invention can include functional utility, whether expressly or implicitly stated herein, or apparent herefrom. However, it is expressly set forth that these benefits are not intended as exclusive. Therefore, any explicit, implicit, or apparent benefit from the disclosure herein is expressly deemed as applicable to the present invention.

According to the present invention, a self-sealing fragrance dispenser can be formed from any one or more materials or combinations of materials, such as one or more of plastic, rubber, wood, metal, a crystalline material, or any other man-made or naturally occurring material, for example and not in limitation, insofar as functionally consistent with the present invention. Further, such a device can be manufactured in any one or more functionally compatible manners, such as through molding, cutting, machining, etc.

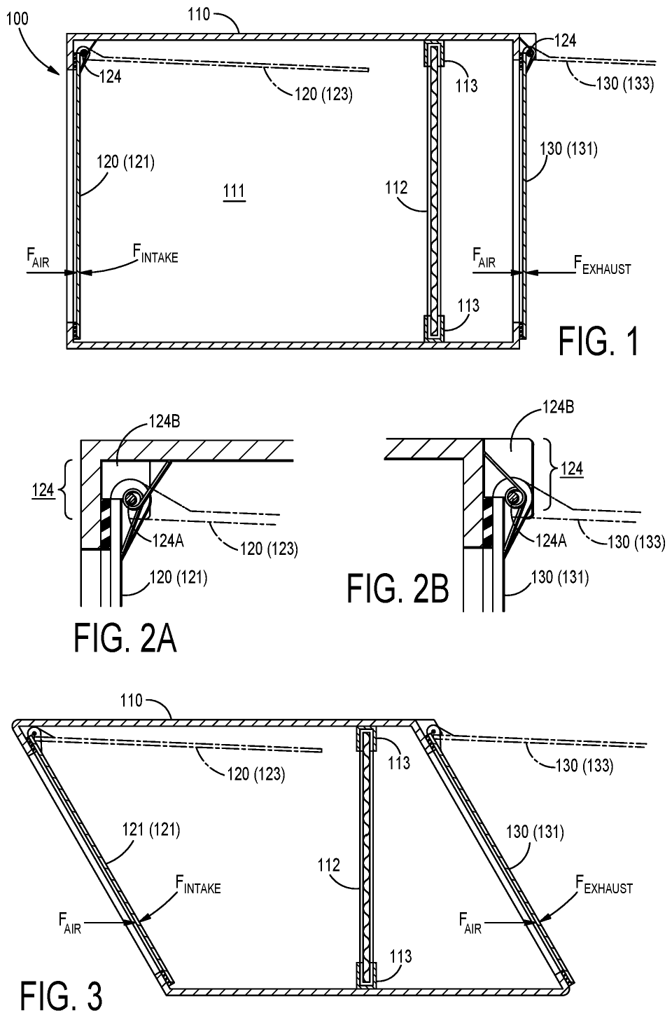

FIG. 1 illustrates an exemplary embodiment of a self-sealing fragrance dispenser for positioning within an air channel (such as an air duct, for example and not in limitation) having active and inactive air flow states.

As illustratively shown, such a dispenser 100 can include a main body 110 having an intake door 120 and an exhaust door 130.

In an exemplary aspect of the present invention, main body 110 can be formed of any one or more non-porous materials defining a cavity 111 configured to encapsulate a fragrance medium 112. As illustratively shown, main body 110 can be provided with a parallelepiped shape; however, the main body can be provided with any desired shape desired insofar as functionally consistent with the invention.

In another exemplary aspect, fragrance medium 112 can be provided as solid, liquid, or gas, with or without an intermediate material (such as a fibrous material with a fragrance composition of matter infused therewith, a structure containing a fragrance medium, etc., for example and not in limitation). As further illustratively shown, fragrance medium 112 can be provided as a fibrous material with a fragrance-based composition of matter infused therewith, for example and not in limitation. As further illustrated, main body 110 can optionally include one or more retention structures 113 to positionally maintain fragrance medium 112 within cavity 111. Notably, retention structures 113 can be provided as rails 113 as illustratively shown, however, can be provided as any functionally compatible structure desired, such as one or more clips, magnets, snaps, etc.

In additional exemplary aspects, intake door 120 can be moveably engaged with main body 110, and can move between intake door closed position 121 and an intake door open position 123 that exposes cavity 111. Likewise, exhaust door 130 can be moveably engaged with main body 110, and can move between an exhaust door closed position 131 and an exhaust door open position 133 that exposes cavity 111.

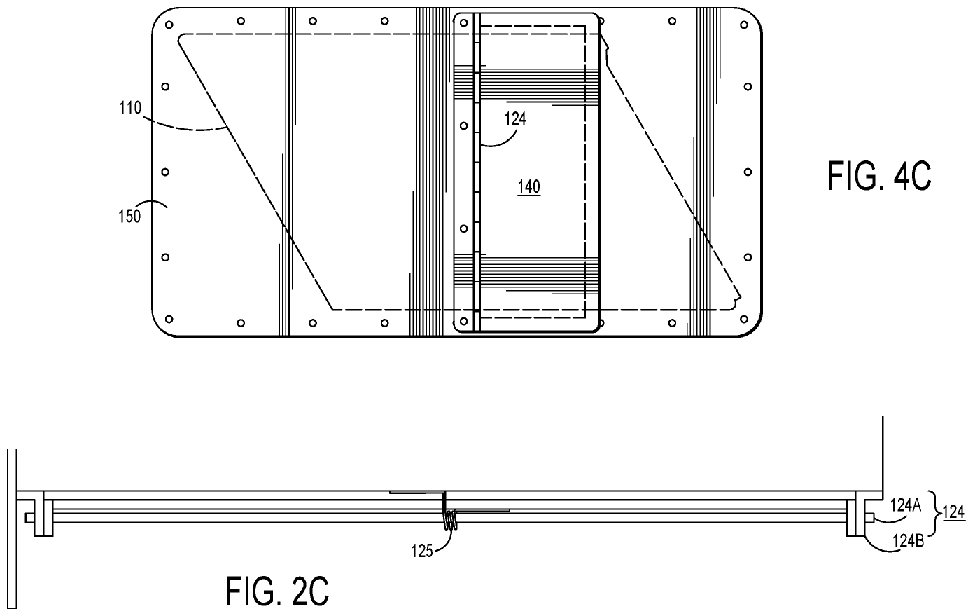

In an exemplary aspect, moveable engagement of intake and exhaust doors 120, 130 can be effectuated via a hinge structure 124, which can be provided as one or more structures. As illustratively shown in FIGS. 2A-2C, hinge structure 124 can be provided as a pivot rod 124a and at least one bracket 124b. Notably, any type of hinge structure, including a living hinge, can be utilized insofar as functionally compatible with the present invention.

In another exemplary aspect, intake and exhaust doors 120, 130 can be respectively biased in intake and exhaust door closed positions 121, 131 via respective intake and exhaust door bias forces F_{INTAKE}, F_{EXHAUST}, which can be provided via mechanical and/or gravitational means. For example and not in limitation, as illustrated in FIGS. 2A and 2B, intake and exhaust door bias forces F_{INTAKE}, F_{EXHAUST} can be provided a spring 125; and as illustrated in FIG. 3, such forces can be provided via gravity, and notably, main body 110 can optionally be skewed to a facilitate the effectively a gravity-based bias force.

In operation, with dispenser 100 positioned within an air channel with an inactive air flow state, intake and exhaust doors 120, 130 can respectively be in closed positions 123, 133 via bias forces F_{INTAKE}, F_{EXHAUST}, which can sealably enclose fragrance medium 112 within cavity 111. Accordingly, when so sealed, the effective lifespan of fragrance medium 112 can be advantageously extended or otherwise improved. It should be noted that such a seal need not be to an exceeding high degree, and can include slight leaking of air. Further, such a seal can be improved or otherwise facilitated an optional gasket with intake door 120, exhaust door 130, or access door 140 (infra). When the air channel is in an active air flow state, a pressure force (F_{AIR}) from the air flow acting against intake and exhaust doors 120, 130 can exceed bias forces F_{INTAKE}, F_{EXHAUST}, so as to move the intake and exhaust doors to their respective open positions 123, 133, which can allow forced air to enter cavity 111 through the intake door, pass over and/or through fragrance medium 112, and carry the fragrance through the exhaust door, such that the fragrance is carried “downwind.”

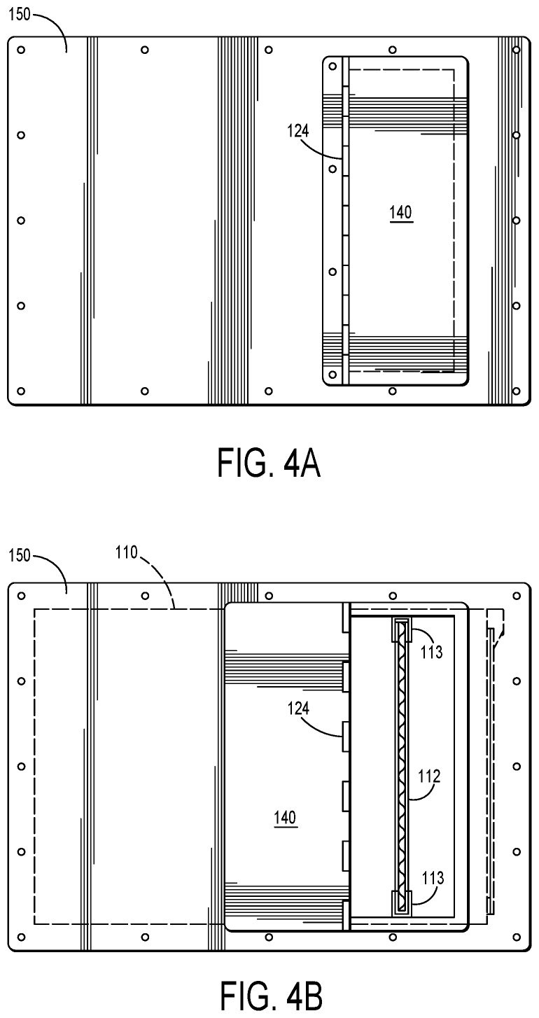

In still another exemplary aspect, main body 110 can optionally include an access door 140 to facilitate optional insertion and/or removal of fragrance medium 112. As illustrated in FIGS. 4A-4C, access door 140 can be moveably engaged, which expressly includes removable attachment, with main body 110 and can be configured to have an access door closed position and an access door open position to allow removable placement of fragrance medium 112 within cavity 111. As with intake and exhaust doors 120, 130, access door 140 can also be provided with a hinge structure 124 to facilitate its transition between open and closed positions. As further illustrated in FIGS. 4A-4C, main body 110 can further include an outer flange or face plate 150 to facilitate attachment of dispenser 100 to an air channel housing, such as an air duct.

It will be apparent to one of ordinary skill in the art that the manner of making and using the claimed invention has been adequately disclosed in the above-written and attached description of the exemplary embodiments and aspects of the present invention.

It should be understood, however, that the invention is not necessarily limited to the specific embodiments, aspects, arrangement, and components shown and described above, but may be susceptible to numerous variations within the scope of the invention.

Therefore, the specification and drawings are to be regarded in an illustrative and enabling, rather than a restrictive, sense.

Accordingly, it will be understood that the above description of the embodiments of the present invention are susceptible to various modifications, changes, and adaptations, and the same are intended to be comprehended within the meaning and range of equivalents apparent to one of ordinary skill in the art.

Therefore, I claim:

1. A self-sealing fragrance dispenser configured for positioning within an air channel having an active and inactive air flow states, said dispenser comprising:
a main body formed of non-porous material defining a cavity configured to surround a fragrance medium;
said main body having an intake door moveably engaged with said main body, the intake door being biased in an intake door closed position by an intake door bias force, and being moveable to an intake door open position that exposes the cavity; and
said main body having an exhaust door moveably engaged with said main body, the exhaust door being biased in an exhaust door closed position by an exhaust door bias force, and being moveable to an exhaust door open position that exposes the cavity;
wherein the intake and exhaust door bias forces are less than respective pressures exerted against the intake and exhaust doors when the air channel is in the active air flow state, and the intake and exhaust door bias forces respectively place the intake and exhaust doors in respective closed intake and closed exhaust door states that seal the amount of fragrance medium within the cavity when the air channel is in the inactive air flow state.

2. The self-sealing fragrance dispenser of claim 1, further comprising the fragrance medium.

3. The self-sealing fragrance dispenser of claim 1, wherein at least one of the intake door bias force and the exhaust door bias force is provided by a bias structure.

4. The self-sealing fragrance dispenser of claim 1, wherein the other of the intake door bias force and the exhaust door bias force is provided by gravity.

5. The self-sealing fragrance dispenser of claim 1, wherein the intake door bias force and the exhaust door bias force are provided by respective bias structures.

6. The self-sealing fragrance dispenser of claim 1, wherein the intake door bias force and the exhaust door bias force are provided by gravity.

7. The self-sealing fragrance dispenser of claim 1, wherein the intake and exhaust doors cooperatively provide an air tight seal around the cavity.

8. The self-sealing fragrance dispenser of claim 1, wherein said main body includes at least one mounting flange configured to mount said main body to a structure defining the air channel.

9. The self-sealing fragrance dispenser of claim 1, wherein said main body includes at least one fixation structure positioned within the cavity and configured to fix the fragrance medium within the cavity.

10. The self-sealing fragrance dispenser of claim 1, wherein said main body further includes an access door moveably engaged with said main body, the access door being configured to have an access door closed position and an access door open position to allow removable placement of the fragrance medium within the cavity.

11. The self-sealing fragrance dispenser of claim 10, further comprising the fragrance medium.

12. The self-sealing fragrance dispenser of claim 10, wherein at least one of the intake door bias force and the exhaust door bias force is provided by a bias structure.

13. The self-sealing fragrance dispenser of claim 10, wherein the other of the intake door bias force and the exhaust door bias force is provided by gravity.

14. The self-sealing fragrance dispenser of claim 10, wherein the intake door bias force and the exhaust door bias force are provided by respective bias structures.

15. The self-sealing fragrance dispenser of claim 10, wherein the intake door bias force and the exhaust door bias force are provided by gravity.

16. The self-sealing fragrance dispenser of claim 10, wherein the access, intake, and exhaust doors cooperatively provide an air tight seal around the cavity.

17. The self-sealing fragrance dispenser of claim 10, wherein said main body includes at least one mounting flange configured to mount said main body to a structure defining the air channel.

18. The self-sealing fragrance dispenser of claim 10, wherein said main body includes at least one fixation structure positioned within the cavity and configured to fix the fragrance medium within the cavity.
